(19) United States
(12) Patent Application Publication
(10) Pub. No.: US 2005/0106120 A1
(43) Pub. Date: May 19, 2005

(54) Polyester containing active drugs and having amino acids in the main chain & comma; and its preparation method

(76) Inventors: Shujun Cheng, Shanghai (CN); Zhiron Tang, Shanghai (CN); Ju Rao, Shanghai (CN)

Correspondence Address: Rabin & Berdo, 1101 14th Street, Suite 500, Washington, DC, 20005, US

(21) Appl. No.: 10/495,328

(22) PCT Filed: Dec. 13, 2001
(86) PCT No.: PCT/CN01/01622

(30) Foreign Application Priority Data
Nov. 12, 2001 (CN) 01132179.2

(51) Int. Cl. C08G 63/48; C08G 63/91; A61K 31/785

(52) U.S. Cl. 424/78.27; 525/54.1; 424/78.37

(57) ABSTRACT

This invention concerns the preparation of certain polyester, which includes amino acid in backbone, and active drug in side chain. It has the general formula as follows: 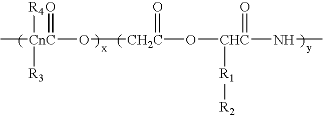
wherein R₁ is aspartic acid, serine, glutamic acid or lysine; R₂ is active drug with reactive group. The preparation of this polymer has three steps: firstly the polymerization of lactide or lactone with derivative of morpholine-2,5-dion, secondly the deprotection, finally the bonding with drug. Through introducing amino acid comprising reactive group to biodegraded polyester, the antigen of the degraded result can be avoided, and it can be absolutely bioabsorbed. Because polymer includes carboxyl, hydroxy and amido, it can have effect directly, or after being degraded. This invention can be used as the coating of medical instruments, and can be prepared for the implant or other regent.

### TECHNIQUE FIELD

This invention concerns the preparation method of certain polyester, which includes amino acid in backbone, and active drug in side chain.

### BACKGROUND TECHNIQUE

With the development of modern iatrology, more and more new cure concepts and correlative medical instruments have been brought up. However, as the biomaterials for medical instruments, there is a basic common gender, that is biocompatibility. Because biodegraded polyesters, for example poly-lactic acid (PLA), poly-glycolic acid (PGA), polycaprolactone (PCL), and copolymer all have excellent biocompatibility and biodegradation, which made them the hotspot in the research of biomaterials in recent years. Polylactide has been authorized for medical surgery seam and injection microcapsule, microball and implant materials by U.S. Food and Drug Administration (FDA). But because of the chemical structure of these polyester, they have a restriction in their performances, that is lack of reactive group.

Drug targeting and continuate release are one of the most appealing hotspots of polymer science in medical field.

Targeting supply drug can be carried through vessel, drug stent or specified antibody. There are two ways of controlled drug release. They are diffuse and degrade release. There are two methods for the combination of drug and polymer, they are physical blending and chemical bonding. Because the drug release of chemical bonding is stabler and affects much more time, it attracts more special attention, and some have been authorized for clinic.

A poly (ethylene oxide) grafting polyurethane, PEO-g-PU has been described in U.S. Pat. No. 5,855,618, which bonds with heparin to obtain a medico-macromolecule. But it has the following weaknesses:
1) The biocompatibility of PU or PEO is not ideal in vivo, and the degraded result is possibly noxious.
2) The end group of PEO is hydroxyl, that only can react with carboxyl, if you want to bond with drug with amido or hydroxyl, you must use diisocyanate, which is virulent, and it takes much more reaction steps.

Therefore, this invention supplies a method to prepare a biodegradable nontoxic medico-macromolecule, which is certainly of great significance.

### DESCRIPTION OF THE INVENTION

One of the technological problems in this invention is how to supply a kind of polyester, which has active drug in side chain and amino acid in backbone, in order to overcome the shortcomings, such as not so good biocompatibility in vivo, the possible noxious degrade result, only reacting with carboxyl, needy of virulent diisocyanate as bridge and much more reaction steps.

The other technological problem in this invention is how to supply a preparation method for the polyester having amino acid and active drug.

The medico-macromolecule in this invention has the following general formula: 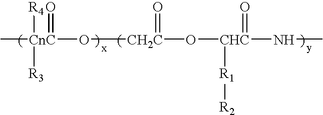
wherein R₁ is either CH₂OH.CH₂COOH.CH₂CH₂COOH or CH₂CH₂CH₂CH₂NH₂, which are usually called serine, aspartic acid, glutamic acid and lysine;
R₂ is drug having reactive group, that is, there are hydroxyl, amido and carboxyl in their formulas;
R₃, R₄ is the side groups of polyester: —H or —CH₃;
α-hydroxide, n=1; β-hydroxide, n=2; γ-hydroxide, n=3; δ-hydroxide, n=4; ε-hydroxide, n=5;
X is polymerization degree, from 1 to 3000, X is polymerization degree, from 1 to 1000;

According to the open structure, this polymer has two parts, one is macromoleculer material, and the other is reactive drug.

The mole ratio of amino acid is 1 to 49 percent, which can be analyzed by ¹H NMR; The molecular weight of macromoleculer material is from 500 to 200000, and the dispersity is from 1.0-3.6, which can be characterized by Gel Permeation Chromatography (GPC).

The above polymer is synthesized within the following steps:

(1) Polymerization

A certain amount of lactide or lactone and derivative of morpholine-dione of lysine, aspartic acid, glutamic acid, serine are put into the reaction tube. And a certain amount of stannous octoate was added into the mixture. The tube was filled with dry N₂ and heated to 120-170° C. for 1-10 hours. The mole ration of lactide or lactone: derivative of morpholine-2,5-dione having amino acid: catalyst is 1: 0.01˜50: 0.0002˜0.002.

(2) Deprotection

The resulting polymer is deprotected, Pd/C or HBr/Hac as catalyst, at 10 to 35° C. for 24 to 80 hours. Biodegraded polyester having reactive side group is obtained.

(3) Bonding

The deprotected polymer and drug are dissolved in solvent, N,N′-dicyclohexyl carbodiimide(DCC) as catalyst, and the biodegraded medico-macromoleculis will be obtained. The reaction temperature is 0 to 5° C., and the reaction time is for 18 to 80 hours. The solvent is one of tetrahydrofuran, chloroform and tetrahydrofuran/H₂O.

### THE SIGNIFICANCE OF THE INVENTION

It is known to all that α-amino acid is the result of poly-amino acid. There are many functional groups in natural amino acid. Although α-amino acid is nontoxic, the degraded oligomer has antigenicity. To avoid the antigenicity caused by degraded result, natural amino acid is randomly introduced into the biodegraded polyester.

Simultaneously the degraded results can be absorbed absolutely. The novel polyester has carboxyl, hydroxyl or amido. As long as the drug has rboxyl, hydroxyl or amido, it can bond with polyester. The obtained medico-macromolecule can work directly, in degradation process as well as after degradation, to effectively restrain the inflammation caused by material.

This invention can be used as coating of medical instruments. It improves the instrument biocompatibility while takes specific drug, which consequently can reach the aim of targeting continually supplying drug. It also can be made into implant or other regent.

The preparation of the polyester of the present invention will be illustrated by the following examples.

### EXAMPLE 1

### Preparation of Poly (lactic acid-co-glycolic acid-co-glutamic Acid)

0.1 mol L-lactide and 0.05 mol derivative of morpholine-2,5-dion with benzyloxycarbonyl glutamic acid are put into a tube with a dry stirring bar. The tube is connected to the Schlerk line, where exhausting-refilling with dry N₂ is replaced for 3 times and put into oil at 160° C. After the mixture is melted, 1 ml 0.02 g/ml solution of stannous octoate in dry chloroform is added into the liquid, and removes the chloroform under vacuum. The tube is heated at 160° C. for 5 h. When the polymerization is finished. The tube is allowed to be cooled to room temperature and get broken. The resulting product is dissolved in chloroform and drops into excess ethyl alcohol. The precipitate is filtered and dried in vacuum at 56° C. for 6 h. The polymer obtained above is dissolved in 20 ml chloroform, Pd/C as catalyst. With vigorous stirring, hydrogengas is bubbled through the suspension for 40 h. The result polymer is poly (lactic acid-co-glycolic acid-co-glutamic acid). The molecular weight is 50000 determined by GPC. The mole ratio of amino acid is 17%.

### EXAMPLE 2

### Preparation of Poly (lactic acid-co-glycolic acid-co-aspartic Acid) Bonding Heparin

The polymer is prepared as described in example 1 but with aspartic acid replacing glutamic acid. The molecular weight is 50000, and mole ratio of amino acid is 17%. Heparin having amido and carboxyl is dissolved in THF/H2O(V_{T}/V_{H}=1:1). 0.5 g heparin sodium dissolves in H₂O, adjusting the PH value to about 4 by diluted acid. With vigorous stirring, the above solutions are put together, and same mole dicyclohexylcarbodiimide (DCC) is put into the mixture and reacts for 24 hours under 4° C. After removing the solvent under vacuum, the result polymer is dissolved in amount of chloroform and indiscerptibility is filtrated, dropped into excess petroleum ether. The precipitate was filtered and dried in vacuum at room temperature for 24 hours. The obtained polymer is heparinized polymer.

### EXAMPLE 3

### Preparation of Poly (lactic acid-co-glycolic acid-co-aspartic Acid) Bonding Penicillium

Penicillium comprises of carboxyl and amido. 2 g poly (lactic acid-co-glycolic acid-co-aspartic acid) described in example 2 and 1 g penicillium are dissolved in chloroform, and same mole DCC is put in. The solvent is vigorous stirring under 4° C. for 24 hours, the result polymer is dropped into excess petroleum ether. The precipitate was filtered and dried in vacuum at room temperature for 24 hours. The obtained polymer is medico-macromolecule.

### EXAMPLE 4

### Preparation of Poly (lactic acid-co-glycolic acid-co-glutamic Acid) Bonding Heparin

3 g poly (lactic acid-co-glycolic acid-co-glutamic acid) described in example 1 dissolves in THF/H₂O, and 0.5 g heparin sodium dissolves in H₂O, adjusting the PH value to about 4 by diluted acid. With vigorous stirring, the above solutions are put together, and same mole dicyclohexylcarbodiimide (DCC) is put into the mixture and reacts for 24 hours under 4° C. After removing the solvent under vacuum, the result polymer dissolves in amount of chloroform and indiscerptibility is filtrated, dropped into excess petroleum ether. The precipitate is filtered and dried in vacuum at room temperature for 24 hours. The obtained polymer is medico-macromolecule.

### EXAMPLE 5

### Preparation of Poly (lactic acid-co-glycolic acid-co-glutamic Acid) Bonding Penicillium

Penicillium comprises of carboxyl and amido. 2 g poly (lactic acid-co-glycolic acid-co-glutamic acid) described in example 1 and 1 g penicillium are dissolved in chloroform, and same mole DCC is put in. The solvent is vigorously stirred under 4° C. for 24 hours, the result polymer is dropped into excess petroleum ether. The precipitate was filtered and dried in vacuum at room temperature for 24 hours. The obtained polymer is medico-macromolecule.

### EXAMPLE 6

### Preparation of Poly (lactic acid-co-glycolic acid-co-serine) Bonding Aspirin

The polymer is prepared as described in example 1 but with serine replacing glutamic acid. Penicillium comprises of carboxyl and amido. 2 g poly (lactic acid-co-glycolic acid-co-serine) and 1.5 g penicillium are dissolved in chloroform, and same mole DCC is put in. The solvent is vigorous stirred under 4° C. for 24 hours, and the result polymer is dropped into excess petroleum ether. The precipitate is filtered and dried in vacuum at room temperature for 24 hours. The obtained polymer is medico-macromolecule.

### EXAMPLE 7

### Preparation of Poly (lactic acid-co-glycolic acid-co-serine) Bonding Penicillium

Penicillium comprises of carboxyl and amido. 2 g poly (lactic acid-co-glycolic acid-co-serine) described in example 6 and 1 g penicillium are dissolved in chloroform, and same mole DCC is put in. The solvent is vigorously stirred under 4° C. for 24 hours, and the result polymer is dropped into excess petroleum ether. The precipitate is filtered and dried in vacuum at room temperature for 24 hours. The obtained polymer is medico-macromolecule.

### EXAMPLE 8

### Preparation of Poly (lactic acid-co-glycolic acid-co-serine) Bonding Heparin

3 g poly (lactic acid-co-glycolic acid-co-serine) described in example 6 is dissolved in THF/H₂O, and 0.5 g heparin sodium dissolves in H₂O, adjusted the PH value to about 4 by diluted acid. With vigorous stirring, the above solutions are put together, and same mole dicyclohexylcarbodiimide (DCC) is put into the mixture and reacts for 24 hours under 4° C. After removing the solvent under vacuum, the result polymer is dissolved in amount of chloroform and indiscerptibility is filtrated, dropped into excess petroleum ether. The precipitate is filtered and dried in vacuum at room temperature for 24 hours. The obtained polymer is medico-macromolecule.

### EXAMPLE 9

### Preparation of Poly (lactic acid-co-glycolic acid-co-lysine) Bonding Penicillium

The polymer is prepared as described in example 1 but with lysine replacing glutamic acid. Penicillium comprises of carboxyl and amido. 2 g poly (lactic acid-co-glycolic acid-co-lysine) and 1 g penicillium are dissolved in chloroform, and same mole DCC is put in. The solvent is vigorously stirred under 4° C. for 24 hours, the result polymer is dropped into excess petroleum ether. The precipitate is filtered and dried in vacuum at room temperature for 24 hours. The obtained polymer is medico-macromolecule.

### EXAMPLE 10

### Preparation of Poly (lactic acid-co-glycolic acid-co-lysine) Bonding Heparin

3 g poly (lactic acid-co-glycolic acid-co-lysine) described in example 9 is dissolved in THF/H₂O, and 0.5 g heparin sodium dissolves in H₂O, adjusted the PH value to about 4 by diluted acid. With vigorous stirring, the above solutions are put together, and same mole dicyclohexylcarbodiimide (DCC) is put into the mixture and reacts for 24 hours under 4° C. After removing the solvent under vacuum, the result polymer is dissolved in amount of chloroform and indiscerptibility is filtrated, dropped into excess petroleum ether. The precipitate is filtered and dried in vacuum at room temperature for 24 hours. The obtained polymer is medico-macromolecule.

### EXAMPLE 11

### Preparation of Poly (lactic acid-co-glycolic acid-co-lysine) Bonding Aspirin

2 g poly (lactic acid-co-glycolic acid-co-lysine) described in example 9 and 1.5 g aspirin are dissolved in chloroform, and same mole DCC is put in. The solvent is vigorously stirred under 4° C. for 24 hours, the result polymer is dropped into excess petroleum ether. The precipitate is filtered and dried in vacuum at room temperature for 24 hours. The obtained polymer is medico-macromolecule.

1. A polyester, comprising of active drug and amino acid in backbone, has the general formula a follows: 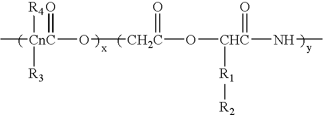
wherein x and y are integers, from 1-3000.
R₁ is either CH₂OH.CH₂COOH.CH₂CH₂COOH or CH₂CH₂CH₂CH₂NH₂;
R₂ is active drug; R₃ and R₄ are the side chains of polyester, including —H and —CH₃;
α-hydroxide, n=1; β-hydroxide, n=2; γ-hydroxide, n=3; δ-hydroxide, n=4; ε-hydroxide, n=5;

2. The polyester mentioned in claim 1 includes the polymer and copolymer of α-hydroxide, β-hydroxide, γ-hydroxide, δ-hydroxide, ε-hydroxide.

3. The polyester mentioned in claim 1 wherein R₂ unit has carboxyl, hydroxy or amido.

4. The polyester mentioned in claim 1 wherein amino acid unit comprises 1-49 mol percent.

5. The molecular weight of the polyester mentioned in claim 1 range from 500 to 200000, while the dispersity is from 1.0-3.6.

6. The preparation method of polyester in claim 1 has the following steps:
(1) Polymerization
Lactide or lactone and derivative of morpholine-2,5-dione having amino acid are put into the polymerization tube, stannous octoate as catalyst, and being kept at 50 to 250° C. for 0.5 to 46 hours.
(2) Deprotection
The resulting polymer is deprotected, Pd/C or HBr/Hac as catalyst, kept at 10 to 35° C. for 8 to 80 hours. Biodegraded polyester having reactive side group is obtained.
(3) Bonding
The deprotected polymer and drug are dissolved in solvent, N,N′-dicyclohexyl carbodiimide(DCC) as catalyst, the biodegraded medico-macromoleculis is obtained. The reaction temperature is −15 to 45° C., and the reaction time is 0.5 to 80 hours.

7. The method according to claim 6, wherein the amino acid includes aspartic acid, serine, glutamic acid or lysine.

8. The method according to claim 6, wherein the mole ration of lactide or lactone: derivative of morpholine-2, 5-dione having amino acid: catalyst is 1: 0.01˜50: 0.0002˜0.05 in the step of polymerization.

9. The method according to claim 6, wherein the polymerization is carried under nitrogen.

10. The method according to claim 6, wherein the solvent is one or admixture of tetrahydrofuran, chloroform, chloromethane, chloroethane, ethylene chloride and tetrahydrofuran/H₂O.

11. The preparation method of polyester in claim 2 has the following steps:
(1) Polymerization
Lactide or lactone and derivative of morpholine-2,5-dione having amino acid are put into the polymerization tube, stannous octoate as catalyst, and being kept at 50 to 250° C. for 0.5 to 46 hours.
(2) Deprotection
The resulting polymer is deprotected, Pd/C or HBr/Hac as catalyst, kept at 10 to 35° C. for 8 to 80 hours. Biodegraded polyester having reactive side group is obtained.
(3) Bonding
The deprotected polymer and drug are dissolved in solvent, N,N′-dicyclohexyl carbodiimide(DCC) as catalyst, the biodegraded medico-macromoleculis is obtained. The reaction temperature is −15 to 45° C., and the reaction time is 0.5 to 80 hours.

12. The preparation method of polyester in claim 3 has the following steps:
(1) Polymerization
Lactide or lactone and derivative of morpholine-2,5-dione having amino acid are put into the polymerization tube, stannous octoate as catalyst, and being kept at 50 to 250° C. for 0.5 to 46 hours.
(2) Deprotection
The resulting polymer is deprotected, Pd/C or HBr/Hac as catalyst, kept at 10 to 35° C. for 8 to 80 hours. Biodegraded polyester having reactive side group is obtained.
(3) Bonding
The deprotected polymer and drug are dissolved in solvent, N,N′-dicyclohexyl carbodiimide(DCC) as catalyst, the biodegraded medico-macromoleculis is obtained. The reaction temperature is −15 to 45° C., and the reaction time is 0.5 to 80 hours.

13. The preparation method of polyester in claim 4 has the following steps:
(1) Polymerization
Lactide or lactone and derivative of morpholine-2,5-dione having amino acid are put into the polymerization tube, stannous octoate as catalyst, and being kept at 50 to 250° C. for 0.5 to 46 hours.
(2) Deprotection
The resulting polymer is deprotected, Pd/C or HBr/Hac as catalyst, kept at 10 to 35° C. for 8 to 80 hours. Biodegraded polyester having reactive side group is obtained.
(3) Bonding
The deprotected polymer and drug are dissolved in solvent, N,N′-dicyclohexyl carbodiimide(DCC) as catalyst, the biodegraded medico-macromoleculis is obtained. The reaction temperature is −15 to 45° C., and the reaction time is 0.5 to 80 hours.

14. The method according to claim 11, wherein the amino acid includes aspartic acid, serine, glutamic acid or lysine.

15. The method according to claim 12, wherein the amino acid includes aspartic acid, serine, glutamic acid or lysine.

16. The method according to claim 13, wherein the amino acid includes aspartic acid, serine, glutamic acid or lysine.

17. The method according to claim 11, wherein the mole ration of lactide or lactone:
derivative of morpholine-2,5-dione having amino acid: catalyst is 1: 0.01˜50: 0.0002˜0.05 in the step of polymerization.

18. The method according to claim 12, wherein the mole ration of lactide or lactone:
derivative of morpholine-2,5-dione having amino acid: catalyst is 1: 0.01˜50: 0.0002˜0.05 in the step of polymerization.

19. The method according to claim 13, wherein the mole ration of lactide or lactone:
derivative of morpholine-2,5-dione having amino acid: catalyst is 1: 0.01˜50: 0.0002˜0.05 in the step of polymerization.

20. The method according to claim 11, wherein the polymerization is carried under nitrogen.

21. The method according to claim 12, wherein the polymerization is carried under nitrogen.

22. The method according to claim 13, wherein the polymerization is carried under nitrogen.

23. The method according to claim 11, wherein the solvent is one or admixture of tetrahydrofuran, chloroform, chloromethane, chloroethane, ethylene chloride and tetrahydrofuran/H₂O.

24. The method according to claim 12, wherein the solvent is one or admixture of tetrahydrofuran, chloroform, chloromethane, chloroethane, ethylene chloride and tetrahydrofuran/H₂O.

25. The method according to claim 13, wherein the solvent is one or admixture of tetrahydrofuran, chloroform, chloromethane, chloroethane, ethylene chloride and tetrahydrofuran/H₂O.

26. The polyester mentioned in claim 1, is used as coating of medical instruments, and not only improves the biocompatibility, but also reaches the aim of target continually supplying medicine. This polyester also can be used as implant or other regent.

27. The polyester mentioned in claim 2, is used as coating of medical instruments, and not only improves the biocompatibility, but also reaches the aim of target continually supplying medicine. This polyester also can be used as implant or other regent.

28. The polyester mentioned in claim 3, is used as coating of medical instruments, and not only improves the biocompatibility, but also reaches the aim of target continually supplying medicine. This polyester also can be used as implant or other regent.

29. The polyester mentioned in claim 4, is used as coating of medical instruments, and not only improves the biocompatibility, but also reaches the aim of target continually supplying medicine. This polyester also can be used as implant or other regent.

30. The polyester mentioned in claim 1, can be used as the coating of stent. The release rate of drug can be controlled after implanted into blood vessel, and it will simultaneously degrade absolutely in 6 months.

31. The polyester mentioned in claim 2, can be used as the coating of stent. The release rate of drug can be controlled after implanted into blood vessel, and it will simultaneously degrade absolutely in 6 months.

32. The polyester mentioned in claim 3, can be used as the coating of stent. The release rate of drug can be controlled after implanted into blood vessel, and it will simultaneously degrade absolutely in 6 months.

33. The polyester mentioned in claim 4, can be used as the coating of stent. The release rate of drug can be controlled after implanted into blood vessel, and it will simultaneously degrade absolutely in 6 months.
